(19) United States
(12) Patent Application Publication
(10) Pub. No.: US 2026/0120836 A1
(43) Pub. Date: Apr. 30, 2026

(54) MULTIMODAL AI-GENERATED PERSONALIZED MOTIVATIONAL, WELLNESS, AND EDUCATIONAL CONTENT SYSTEM

(71) Applicant: Iconic Inventions LLC, Oak Park, CA (US)

(72) Inventor: John DEVECKA, Oak Park, CA (US)

(21) Appl. No.: 19/373,505

(22) Filed: Oct. 29, 2025

Related U.S. Application Data
(60) Provisional application No. 63/713,490, filed on Oct. 29, 2024.
(60) Provisional application No. 63/842,606, filed on Jul. 11, 2025.

(51) Int. Cl. G16H 20/00; G16H 40/67

(52) U.S. Cl. CPC G16H 20/00; G16H 40/67

(57) ABSTRACT

A system and method are disclosed for generating personalized motivational, wellness, educational, and entertainment multimedia content using multimodal artificial intelligence. The system constructs an Emotion State Vector (ESV) from user inputs, diaries, biometric data, and context. A Modality Script Generator (MSG) defines time-coded orchestration across music, narration, captions, and visuals. Scripts execute via a Model-Agnostic Adapter Layer (MAAL) across heterogeneous AI engines. A Synchronization Graph (SyncGraph) maintains acceptable inter-modal drift, while a Dynamic Media Formatter (DMF) applies bounded repair policies and packages synchronized outputs with timed metadata. A Policy/Constraint Graph enforces rights and provenance, logged in a Transform Manifest.

### CROSS-REFERENCES

The following applications and materials are incorporated herein by reference, in their entireties, for all purposes: U.S. Provisional Patent Application Serial Nos. 63/713,490 filed Oct. 29, 2024, and 63/842,606, filed Jul. 11, 2025.

### FIELD

This disclosure relates to systems and methods for providing personalized audio and audiovisual content to a user. More specifically, the disclosed embodiments relate to systems and methods for generating personalized motivational, wellness, spiritual, entertainment, and educational content across multiple modalities.

### INTRODUCTION

Existing multimedia platforms that deliver motivational, wellness, or educational content predominantly rely on static, pre-recorded materials. These systems cannot dynamically adapt to a user's emotional state, personal challenges, learning goals, or contextual environment. While some AI-based systems generate music or recommend content based on preferences, such systems typically operate in isolation within a single modality and lack synchronization across modalities.

Furthermore, existing adaptive educational technologies often focus on adjusting difficulty levels of material rather than tailoring delivery style, emotional framing, or multi-modal integration. As a result, current platforms fail to provide deeply personalized experiences that respond, for example, to real-time biometric feedback, contextual factors such as location or activity, and evolving user preferences. This gap limits user engagement and reduces the efficacy of motivational and educational interventions.

### SUMMARY

The present disclosure provides systems, apparatuses, and methods relating to personalized content.

In some examples, a computer-implemented method may include: automatically constructing an emotion state vector (ESV) configured to encode an affective state of a user, wherein the ESV comprises a plurality of dimensions each based on a respective sensed input related to the user; constructing, based on the ESV, a modality script comprising information defining and orchestrating a plurality of media content items; generating the plurality of media content items using one or more generative artificial intelligence (AI) engines; arranging the plurality of media content items into a media stream according to the modality script; and queueing the media stream to enable playback on an electronic device of the user.

In some examples, a system for generating personalized digital content may include: one or more processors; a memory; and a plurality of instructions stored in the memory, wherein the plurality of instructions, when executed by the one or more processors, are configured to: automatically construct an emotion state vector (ESV) configured to encode an affective state of a user, wherein the ESV comprises a plurality of dimensions each based on a respective sensed input related to the user; construct, based on the ESV, a modality script comprising information defining and orchestrating a plurality of media content items; generate the plurality of media content items by communicating with one or more generative artificial intelligence (AI) engines; arrange the plurality of media content items into a media stream according to the modality script; and queue the media stream to enable playback on an electronic device of the user.

A non-transitory computer readable storage medium storing instructions that, when executed, cause one or more processors to: automatically construct an emotion state vector (ESV) configured to encode an affective state of a user, wherein the ESV comprises a plurality of dimensions each based on a respective sensed input related to the user; construct, based on the ESV, a modality script comprising information defining and orchestrating a plurality of media content items; generate the plurality of media content items by communicating with one or more generative artificial intelligence (AI) engines; arrange the plurality of media content items into a media stream according to the modality script; and queue the media stream to enable playback on an electronic device of the user.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a schematic system context diagram showing an illustrative system in accordance with aspects of the present disclosure.

FIG. 2 is a flow chart depicting steps of an illustrative method for providing personalized content according to the present teachings.

FIG. 3 is a component diagram depicting components of an illustrative content personalization system in accordance with aspects of the present disclosure.

FIG. 4 is a schematic diagram depicting components of the system of FIG. 3 with illustrative inputs, outputs, and data flow.

### DETAILED DESCRIPTION

Various aspects and examples of a personalized content system, as well as related methods, are described below and illustrated in the associated drawings. Unless otherwise specified, a personalized content system in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

A “controller” or “electronic controller” includes processing logic programmed with instructions to carry out a controlling function with respect to a control element. For example, an electronic controller may be configured to receive an input signal, compare the input signal to a selected control value or setpoint value, and determine an output signal to a control element (e.g., a motor or actuator) to provide corrective action based on the comparison. In another example, an electronic controller may be configured to interface between a host device (e.g., a desktop computer, a mainframe, etc.) and a peripheral device (e.g., a memory device, an input/output device, etc.) to control and/or monitor input and output signals to and from the peripheral device.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

Disclosed herein is a computer-based architecture providing an adaptive, AI-driven, multimedia streaming service. In general, personalized content systems and methods of the present disclosure may include a multimodal AI system that generates personalized content for a user based on the user's preferences and affective state. In some examples and use cases, this may be described as “Motivational Multimedia as a Service.” A personalization agent of the system ingests a baseline personalization profile (BPP) from the user and/or formulates an emotion state vector (ESV) based on sensed inputs. Together, the BPP and ESV capture a user's emotional and contextual state using various inputs such as structured prompts, diary entries, biometric data, and media uploads. The ESV and BPP are used by the system along with a concept graph to map user goals and challenges to appropriate modalities and framing methods, where appropriate means the modalities and framing methods are either compatible with or prescriptive with respect to one or more of the goals and/or challenges of the user.

Based on the mapping, a modality script generator (MSG) is utilized to create a time-coded orchestration plan or script, defining the roles and directives for different modalities such as music, narration, visuals, and captions. In some examples, different modalities are configured to overlap, such as when a narration plays over a corresponding visual or background instrumental music. The MSG may support predefined templates and may be user-editable, allowing for dynamic switching between segment variants without full regeneration. Modular design enables integration across cloud, edge, and on-device environments.

The system uses a model-agnostic adapter layer (MAAL) to execute the scripts and directives across one or more (e.g., heterogeneous) artificial intelligence (AI) generation engines, while ensuring normalized output. In other words, the MAAL works with one or more AI models or agents to generate the materials that populate the script or orchestration plan produced by the MSG. The materials, referred to as media content items, may be retrieved from an existing database of such materials, and/or may be generated or altered by the AI model(s). A synchronization graph (referred to as the SyncGraph) links elements like beats, phonemes, captions, and video frames, maintaining bounded drift (e.g., to ensure narration matches up with visuals), while a dynamic media formatter (DMF) assembles the final outputs, applying repair policies to ensure synchronization and accessibility.

A policy/constraint graph manages intellectual property rights and provenance, with all events logged in a manifest. The disclosed systems and methods enable dynamic, context-aware generation of synchronized multimedia content tailored to a user's emotional state, preferences, and goals. In some examples, the present disclosure integrates an emotion-preserving translator, gamification features, and/or enterprise compliance layers. Accordingly, the disclosed platform goes beyond existing single-modality systems (e.g., song playlists) by providing a pipeline that ensures real-time multimodal alignment and drift control, exposing a section-level orchestration editor, and integrating licensed media under policy constraints. The system's architecture allows for the incorporation of user-imported media, creative visualization modules, and group progress trackers, all while maintaining synchronization and rights enforcement. It supports various operation modes, including streaming, batch, and hybrid, adapting to user signals in real-time and continuously updating personalization parameters for future sessions.

Technical solutions are disclosed herein for generating adaptive, multimodal, and personalized content. Specifically, the disclosed system/method addresses a technical problem tied to artificial intelligence and multimedia content generation technology and arising in the realm of computers, namely the technical problem of existing multimedia platforms and AI-based systems failing to provide deeply personalized experiences that respond to real-time biometric feedback, contextual factors, and evolving user preferences. The system and method disclosed herein provides an improved solution to this technical problem by employing a Personalization Agent that constructs an Emotion State Vector (ESV) encoding a user's affective and contextual state, combined with a Modality Script Generator (MSG) that defines time-coded roles and directives for various modalities, executed through a Model-Agnostic Adapter Layer (MAAL) across heterogeneous generation engines, and synchronized using a Synchronization Graph (SyncGraph) with bounded drift constraints, further assembled by a Dynamic Media Formatter (DMF) applying repair policies.

Solutions disclosed herein are configured to generate adaptive, multimodal, and personalized content. This means the system aims to create multimedia content that can adjust to an individual's specific needs, preferences, and emotional state, utilizing various forms of media. Known systems often rely on static, pre-recorded material that cannot dynamically adapt, for example, to a user's emotional state, personal challenges, learning goals, or contextual environment. While some AI-based systems can generate music or recommend content based on preferences, they typically operate in isolation within a single modality and lack individualized multimedia content with seamless synchronization across modalities. Furthermore, known adaptive educational technologies often focus on adjusting the difficulty levels of the material rather than tailoring the delivery style, emotional framing, or multi-modal integration. As a result, existing platforms fail to provide deeply personalized emotive-media experiences that respond to real-time biometric feedback, contextual factors, and evolving user preferences. This gap limits user engagement and reduces the efficacy of motivational and educational interventions.

To address these issues, the disclosed systems employ several key components:
Personalization Agent and Emotion State Vector (ESV): The Personalization Agent constructs an Emotion State Vector (ESV) that encodes a user's affective and contextual state. The ESV acts as the system's live control signal, aggregating numerical scores from language-based goal input, AI-coach dialogue, profile preferences, and secondary biometric/contextual sensors. The ESV directly guides every downstream module.
Modality Script Generator (MSG): The ESV is combined with a Modality Script Generator (MSG) that defines time-coded roles and directives for music, narration, visuals, and captions. The MSG produces a time-coded orchestration plan specifying segment roles and directives for music, narration, visuals, and captions. The Modality Script supports predefined templates such as breakout/breakdown interludes, guided-meditation flows, mnemonic explainers, and content magnification transitions.
Model-Agnostic Adapter Layer (MAAL): Scripts and directives are executed through a Model-Agnostic Adapter Layer (MAAL) across heterogeneous generation engines. The MAAL directs tasks to heterogeneous engines and returns normalized artifacts. The MAAL provides standardized input/output adapters so that the orchestration plan (MSG) can execute consistently regardless of which model vendor or library is used.
Synchronization Graph (SyncGraph): Outputs are synchronized using a Synchronization Graph (SyncGraph) that links beats, phonemes, captions, and video frames, with bounded drift constraints (e.g., ≤50 ms). The SyncGraph links beats/bars, phonemes/syllables, caption tokens, video frames, and interaction cues at ≥10 ms resolution.
Dynamic Media Formatter (DMF): A Dynamic Media Formatter (DMF) assembles final audio-only and audiovisual outputs and applies repair policies such as caption retiming, micro tempo-stretching (±1-3%), layer thinning, and frame resampling. The DMF maintains inter-modal drift ≤50 ms and applies bounded repair.

By coordinating these modules, the system enables real-time, personalized, and synchronized content creation that adapts to emotional, physiological, and contextual factors, offering benefits not provided by prior art systems. The system goes beyond static streaming services by integrating real-time personalization, SyncGraph-timed metadata, and manifest-based rights enforcement. The transformational layers are realized by an ESV→MSG→MAAL→SyncGraph→DMF pipeline with numeric timing and repair constraints, policy-governed licensed integrations, translator quality assurance for affect preservation, and manifest-based provenance. This yields concrete machine-level improvements over recommendation-only or unsynchronized generation systems by enforcing deterministic alignment, repair, and compliance at runtime.

The disclosed systems and methods provide an integrated practical application of the principles discussed herein. Specifically, the disclosed systems and methods:
result in an improvement in the functioning of a computer. The system's modular architecture supports deployment across cloud, edge, and on-device environments, enabling both high-throughput streaming services and low-latency local playback.
lead to an improvement to a technology or technical field. The disclosed system enables real-time, personalized, and synchronized content creation that adapts to emotional, physiological, and contextual factors, offering benefits not provided by prior art systems. For example, the present systems and methods may be configured to use cognitive and behavioral methodologies to generate personalized scripts, e.g., for lyrics or narrations. The system integrates an Emotion State Vector (ESV)-conditioned Modality Script (MSG) with a Model-Agnostic Adapter Layer (MAAL), Synchronization Graph (SyncGraph), and Dynamic Media Formatter (DMF) pipeline to enforce real-time multimodal alignment and drift control, which is unlike audio-only prompt generators.
describes a specific manner of generating personalized multimedia content, which provides specific improvements over prior systems and results in an improved personalized multimedia output. The system constructs an Emotion State Vector (ESV), e.g., from user inputs (e.g., regarding goals, challenges, needs, etc.), diaries, biometric data, and contextual metadata. A Modality Script Generator (MSG) produces a time-coded orchestration plan specifying segment roles and directives for music, narration, visuals, and captions. The script executes through a Model-Agnostic Adapter Layer (MAAL) across heterogeneous AI engines. A Synchronization Graph (SyncGraph) enforces bounded drift between modalities, and a Dynamic Media Formatter (DMF) assembles synchronized outputs while applying repair policies. Provenance, licensing, and rights enforcement are managed by a Policy/Constraint Graph and logged in a Transform Manifest.

Accordingly, the disclosed systems and methods apply (or use) the relevant principles in a meaningfully limited way.

Technical issues addressed by the present disclosure include the fact that existing multimedia platforms and AI-based systems fail to provide deeply personalized experiences that respond to real-time biometric feedback, contextual factors, and evolving user preferences. Known systems often rely on static, pre-recorded material or operate in isolation within a single modality, lacking individualized multimedia content with seamless synchronization across modalities. Furthermore, existing adaptive educational technologies primarily focus on adjusting difficulty levels, rather than tailoring delivery style, emotional framing, or multi-modal integration. This limits user engagement and reduces, for example, the efficacy of motivational and educational interventions.

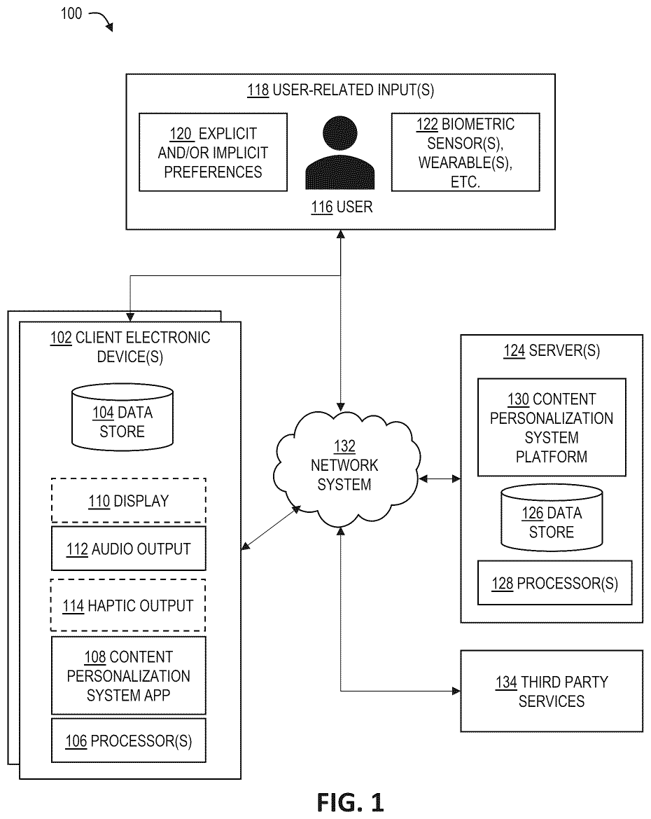

FIG. 1 is a system context diagram depicting an illustrative personalized content system 100 according to the present teachings. In this example, one or more electronic devices 102 (e.g., smart phones, tablet computers, laptop computers, desktop computers, AI-enabled devices, AI-enabled recorders, etc.) include a data store 104, processing logic including one or more processors 106 configured to execute a content personalization system application 108 (AKA an app), and outputs such as a display 110, audio output 112, and in some examples other outputs such as a haptic output 114. In some examples, electronic device 102 may comprise speaker(s), headphones, or ear buds. In some examples, electronic device 102 may comprise an augmented reality (AR) and/or virtual reality (VR) and/or mixed reality (XR) device. For example, electronic device 102 may comprise smart glasses, a VR headset, or AI-enabled AR glasses.

A user 116 utilizes the electronic device to interact with content personalization system app 108, and information relating to user 116 is provided as input(s) 118 to the device and the app. For example, explicit and/or implicit preferences 120 may be provided by the user, as well as one or more sensed metrics 122, such as heart rate or respiration, motion tracking, walking or running cadence, etc., which may be collected by wearable and/or biometric devices of the user.

In a client-server type of setup, one or more server devices 124 (AKA servers or server computers) are in electronic communication with electronic device(s) 102. Server(s) 124 include a data store 126 and one or more processors 128, and may be configured to run a content personalization platform application 130 to coordinate server-side functionality of the overall system. Various functionality may be shared as needed or desired between app 108 on the electronic device and application 130 on the server. In some examples, application 130 functions to keep relevant data synched between the server and the electronic device as well as between several electronic devices if present. Server(s) 124 and/or electronic device(s) 102 may communicate with third party services 134 for functionality such as rights management and compliance.

The various parts and devices of system 100 may communicate by way of a computer network 132, interchangeably termed a network system, a distributed data processing system, or a distributed network. Network 132 may be implemented as one or more of different types of networks. For example, network 132 may include an intranet, a local area network (LAN), a wide area network (WAN), or a personal area network (PAN). In some examples, network 132 includes the Internet, with network 132 representing a worldwide collection of networks and gateways that use the transmission control protocol/Internet protocol (TCP/IP) suite of protocols to communicate with one another. In some examples, network 132 may be referred to as a “cloud.”

Aspects of the personalized content systems disclosed herein may be embodied as a computer method, computer system, or computer program product. Accordingly, aspects of the personalized content system may take the form of an entirely hardware embodiment, an entirely software embodiment (including firmware, resident software, micro-code, AI models or software running locally (AKA on the edge), and the like), or an embodiment combining software and hardware aspects, all of which may generally be referred to herein as a “circuit,” “module,” or “system.” Furthermore, aspects of the personalized content system may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the personalized content system may be written in one or any combination of programming languages, including an object-oriented programming language (such as Java, C++), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the personalized content system may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the personalized content system. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

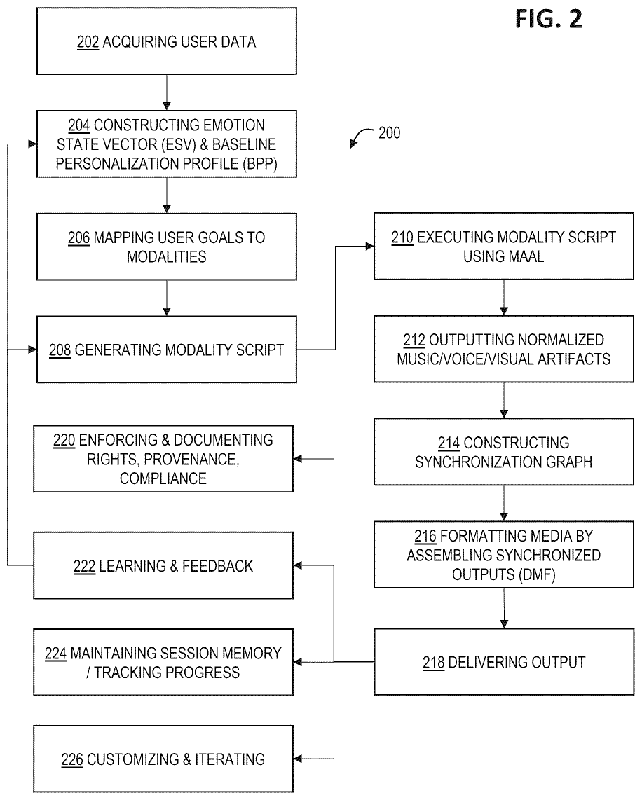

FIG. 2 is a flow chart depicting steps of an illustrative method 200 executable by system 100 described above. The overall method will now be described, with further detail provided in Sections A-D below.

As depicted in FIG. 2, step 202 of method 200 includes acquiring or receiving user data relating, for example, to the user's goals, challenges, preferences, and affective state. This data may include implicit preferences, explicit preferences, and biometric data. Step 202 may include collecting structured prompts, diary-like free text, AI-coach interviews, system-administered surveys, personality or psychometric assessments, and self-reported challenges or goals. Step 202 may involve gathering media uploads such as photos, videos, text, educational content, or personal audio, which may be tagged or annotated by the user. Implicit or sensed inputs, such as biometric and voice-biometric signals (e.g., heart rate, skin conductance, stress, step count, pitch, jitter, prosody, tone variation) and contextual data (e.g., time, location, weather, social activity), may be captured. The system may utilize one or more wearable devices with integrated biometric sensors and/or computer vision-based sensors to collect physiological data, such as heart rate, stress levels, and skin conductance. In some examples, AI-driven cameras and microphones are utilized to track user performance and provide contextual activity and/or environmental data.

Step 204 of method 200 includes constructing a baseline personalization profile (BPP) and emotion state vector (ESV) based on the user data acquired in step 202. The BPP is configured to store stable traits such as preferences, goals, long-term challenges, and preferred persona style, while the ESV is configured to function as a transient, multi-dimensional vector representing the user's momentary emotional and contextual state. The ESV, which combines factors such as linguistic data, biometric data, and contextual data, functions as a compact machine-readable “emotional fingerprint” of the user's momentary state. The BPP initializes ESV priors and may be configured to indicate an initial modality script template selection in step 208.

Step 206 of method 200 includes mapping the user's affective state and profile, based on the BPP and ESV, to one or more modalities using a mapping process. This may include mapping user goals to motivational genres and instructional methods using a Concept Graph. The Concept Graph maps goals/challenges to modalities and framing methods, such as Cognitive Behavioral Therapy (CBT) or the Hero's Journey. The ESV guides every downstream module. For example, in the Modality Script Generator (MSG), the ESV selects or rewrites the narrative path, determines pacing, and chooses framing modalities. In some examples, the modality script may be reviewed and edited by the user for further customization.

Step 208 of method 200 includes generating a modality script based on the modalities output by step 206. This may include producing a time-coded orchestration plan with segment roles, directives, and bindings, using the Modality Script Generator (MSG). The modality script specifies segment roles and directives for script, music, voice, visuals, and captions with bindings to licensed or user-provided resources. The Modality Script Generator supports predefined templates such as breakout/breakdown interludes, guided-meditation flows, mnemonic explainers, and content magnification transitions. In some examples, the modality script is user-visible and section-editable through an interface and/or using natural-language prompts.

Step 210 of method 200 includes executing the modality script using a Model-Agnostic Adapter Layer (MAAL) configured to normalize the outputs from different AI generators into a unified data structure for synchronization. This may include directing tasks to heterogeneous engines, such as text-to-speech (TTS) including script, narration, AI voice and/or vocal cloning, music generation (original, licensed, or remixed), and visual generation (GAN/diffusion/AR/VR). The MAAL provides standardized input/output adapters so that the orchestration plan (i.e., modality script) can execute consistently regardless of which model vendor or library is used. The MAAL receives time-coded segment directives from the modality script, and is configured to dispatch tasks to one or more different AI engines as needed. AI engines may include any suitable generative AI solution and any suitable AI model, such as those currently commercially available under the names ChatGPT, Claude, Gemini, Copilot, and Grok, as well as any suitable open-source solution and/or purpose-build AI application for generating music, voice, etc.

Step 212 of method 200 includes outputting normalized media artifacts which include phoneme timings for voice, beat grids for music, and presentation timestamps for video frames. This may include converting model-specific outputs into a normalized artifact schema used by the Synchronization Graph (SyncGraph). Normalized artifacts might look like: Audio: {wav_file, phoneme_timestamps}; Music: {stems[ ], beat_grid[ ], bpm}; Video: {frames[ ], frame_pts[ ], resolution}. This normalization ensures the SyncGraph (see step 214) can measure alignment and drift across modalities.

Step 214 of method 200 includes constructing the synchronization graph (SyncGraph), which may include linking beats, phonemes, caption tokens, and video frames with computed alignment confidence. The SyncGraph is a representation of the alignment between different modalities, ensuring that audio, lyrics, visuals, and interaction cues are synchronized in real time. The SyncGraph links beats/bars, phonemes/syllables, caption tokens, video frames, and interaction cues at a high resolution, such as 10 ms resolution. The synchronization graph enforces bounded drift (e.g., under 50 ms) between modalities. A lightweight SyncGraph (e.g., with 20 ms resolution) may be used in low-resource deployments, and may buffer media (e.g., up to 10 seconds worth of media).

Step 216 of method 200 includes formatting media by assembling synchronized outputs using a dynamic media formatter (DMF) configured to maintain inter-modal drift within acceptable limits. This may include applying bounded repair policies to maintain synchronization. In some examples, the DMF assembles final audio-only and audiovisual outputs. This involves applying repair policies such as caption retiming, micro tempo-stretching (e.g., ±1-3%), layer thinning, and/or frame resampling. The DMF maintains inter-modal drift at an acceptable level (e.g., ≤50 ms) and applies bounded repair based on alignment-confidence thresholds. The DMF may insert supportive captions or overlays at emotional peaks. The DMF may apply safe-area cropping and accessibility color/contrast transforms. The DMF preserves SyncGraph timing and manifest identifiers for provenance downstream.

Step 218 of method 200 includes delivering the output to the user in the form of a synchronized streaming package or export. This may include packaging timed metadata (e.g., Web Video Text Tracks (WebVTT), SubRip Subtitles (SRT), beat markers, etc.). A streaming service layer manages distribution, recommendation, and monetization of generated content. Any suitable and/or user-selected delivery mode may be utilized, such as micro-bursts (e.g., ≤30 s), guided journeys (e.g., 3-10 min), and episodic series. The outputs may be packaged with SyncGraph metadata for adaptive bitrate (ABR) streaming. The system may support private, community/remix, and branded/licensed libraries. The client runtime may be configured to use jitter buffers and/or micro time-stretch to maintain acceptable levels of drift across device classes. ABR adaptation (e.g., HLS/DASH) plus buffering management may be utilized to preserve sync under variable networks.

Step 220 of method 200 includes enforcing and documenting rights, provenance, and compliance. This may include using a Policy/Constraint Graph to manage provenance, licensing, user constraints, and/or rights enforcement. The Policy/Constraint Graph ensures licensed and AI-generated elements remain compliant. The system may also employ verification filters. A Transform Manifest logs drift violations and licensing events, ensuring attribution, auditability, and royalty allocation. The Policy Graph validates transforms before export (e.g., overlay-only rules). Manifest entries log operation type, segment IDs, timecodes, editor ID, and license tokens. In some examples, immutable blockchain logging is utilized to ensure attribution, auditability, and royalty allocation. Rights enforcement is integrated with the streaming service layer. License tokens/timecodes are checked against usage constraints; formats are standardized (e.g., 44.1 kHz stereo) to ensure compatibility.

Step 222 of method 200 includes learning and providing feedback based on user activity, such as ratings, saves, edits, replays, dwell time, and skips. This may include updating personalization and MSG templates via reinforcement and statistical trend detection. Section-level interactions may be utilized to produce impact weights, e.g., with time decay. Updates to ESV priors and templates are recorded to the Transform Manifest for audit and A/B evaluation. In some examples, a recommendation module is configured to auto-select the next media length/type and adjust MSG templates accordingly. In this manner, the system may directly influence orchestration and personalization.

Step 224 of method 200 includes maintaining session memory and tracking progress. This means that user or cohort embeddings and episode descriptors are preserved across serialized sessions (e.g., training or therapy). This may include using a Session Memory Graph, which is a longitudinal store. Retention is enforced through automated purge scripts (e.g., 90-day cycle) with logged deletions for compliance. A gamification subsystem ties progress tracking and achievements directly to motivational and educational media sets, tests, and surveys, feeding adaptive personalization. Metrics may include completion ratio, streak days, impact score, mastery score, and adherence variance. Badges may be awarded and goal transitions logged in the Transform Manifest.

Step 226 of method 200 includes customizing and iterating the Modality Script Generator (MSG) and/or Emotional State Vector (ESV). This may include enabling localized user edits without regenerating the entire work. In some examples, functionality is included to facilitate users appending journal snippets linked to segments. The ESV is continuously updated in real time as the user interacts, allowing the system to re-score segments or generate new variants dynamically. A live, vectorized BPP programmatically shapes MSG and ESV, unlike static preference toggles. The system provides a section-level orchestration editor for narration, music, visuals, and captions, enabling localized edits without regenerating the entire work.

### Examples, Components, and Alternatives

The following sections describe selected aspects of illustrative personalized content systems as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Personalized Content System

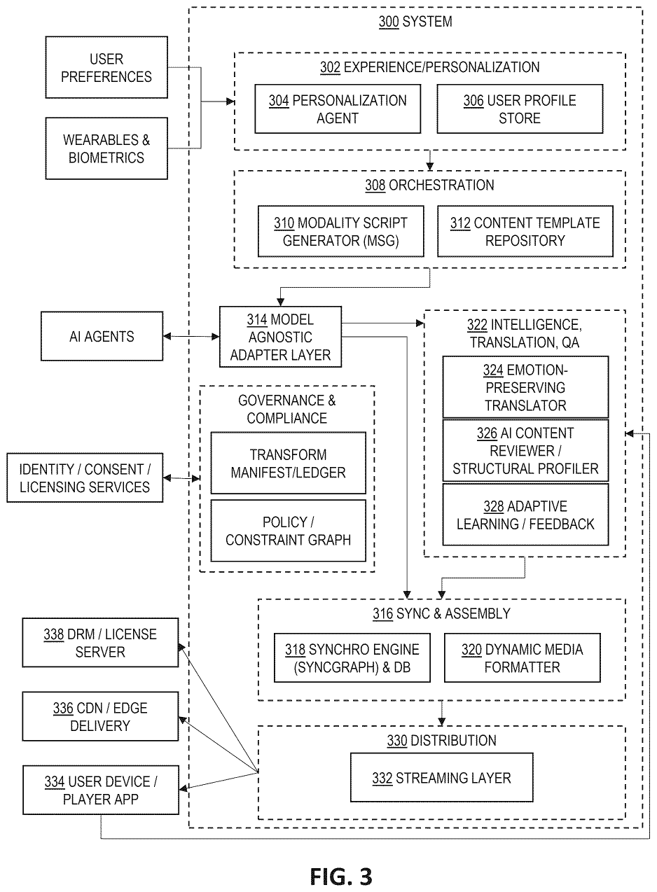

As shown in FIG. 3, this section describes an illustrative personalized content system 300 which is an example of the systems described above.

FIG. 3 is a component diagram depicting the building blocks of personalized content system 300 and their interrelationships. A personalization module 302 is configured to generate and store one or more numerical vectors representing the user's affective state, context, and preferences. Module 302 includes a personalization agent 304 and a user profile store 306. Personalization agent 304 is a software component configured to construct an Emotion State Vector (ESV) by encoding the user's affective and contextual state based on sensed information associated with the user. The ESV serves as the central control signal for the system: a live, numerical snapshot of the user's emotional and physiological state, vectorizing the user's qualitative affect to drive multimodal generation downstream. User profile store 306 includes any suitable data store configured to save stable user traits such as preferences, goals, long-term challenges, and persona style. This may be saved as a Baseline Personalization Profile (BPP). Together, the personalization agent's ESV and the user profile store's BPP create a comprehensive understanding of the user, combining transient emotional states with stable personal traits. In general, the BPP stores stable traits, e.g., based on explicit and implicit user preferences, and the ESV represents momentary state, based on sensed (e.g., biometric, computer vision) inputs from the user (e.g., from wearable devices or sensors).

The ESV is configured to enable the system to understand and react to a user's emotional state, and may be understood as a live numerical snapshot of the user's emotional and contextual state. In particular, the ESV is a multi-dimensional vector made up of several individual values, each representing a different aspect of the user's state. Each of these values is normalized to have a value from 0 to 1. The ESV may include one or more of the following dimensions:
Arousal
   Represents the user's overall activation or energy level.
   Can be derived from physiological signals such as heart rate, indicators of stress, or motion.
   May be calculated by normalizing the sum of the Z-score of the user's heart rate (HR) and a weighted measure of speech intensity. For example, arousal=normalize (zscore(HR)+0.5*speech_intensity)
Valence
   Captures the positive or negative sentiment of the user.
   May be derived by applying a sigmoid function to a sentiment score obtained from natural language processing (NLP). For example, valence=sigmoid(sentiment_score)
Focus
   The stability of the user's attention, e.g., measured through gaze tracking or speech patterns.
   May be calculated as the inverse variance of the user's gaze vector stability. For example, focus=inverse_variance(gaze_vector_stability)
Fatigue
   Reflects the user's level of tiredness, e.g., inferred from blink rate or vocal softness.
   May be calculated as one minus the normalized energy level. For example, fatigue=1−normalize(energy_level)
Confidence
   The system's assessment of the user's confidence level, e.g., based on tone of voice and word choices.
   May be determined by applying a softmax function to the sum of the probability of confidence derived from the user's voice and a weighted ratio of positive words used. For example, confidence=softmax(prob_confident_from_voice+0.4*positive_word_ratio)
Readiness
   Calculated composite value, e.g., a weighted combination of arousal and confidence. For example, readiness=0.6*arousal+0.4*confidence.

The ESV considers linguistic data, biometric data, and contextual/historical data. Each dimension is computed as a weighted, normalized scalar value.

The system uses these formulas to translate raw data into meaningful emotional metrics. Natural language processing and sentiment classifiers extract emotional metrics from user statements. Vocal prosody is analyzed for arousal and stress. Real-time wearable inputs like heart rate and skin conductance act as corroborating data. Contextual data like location, time, activity, device being used, etc. can also influence the ESV.

In general, the ESV is not static but is continuously updated in real time as the user interacts with the system. The ESV vector is configured to update at a selected rate (e.g., 2-10 times per second) using exponential smoothing. In some examples, the formula for this update is as follows for any given dimension:

$$ESV\_Dimension \_t = ( α * input\_t ) + ( ( 1 - α ) * ESV\_Dimension \_t - 1 )$$

Where:
ESV_Dimension_t is the ESV dimension's value at the current time.
input_t is the new input value at the current time.
α is a smoothing factor (typically around 0.3) that controls how responsive the ESV is to new inputs.
ESV_Dimension _t−1 is the ESV dimension's value at the previous time step.

A hysteresis threshold (e.g., approximately 0.1) may be utilized to prevent overreaction to noise in the input data. This helps to smooth out fluctuations and ensure the ESV remains stable. Dimensions may be weighted by a weight vector (e.g., [0.25,0.25,0.15,0.10,0.10,0.15]) configured to tune relative importance of the dimensions depending on the use case (e.g., education vs. fitness).

An orchestration module 308 receives the ESV and BPP and generates a modality script using a modality script generator (MSG) 310 and a content template repository 312. MSG 310 is a software component configured to produce a time-coded orchestration plan that specifies segment roles and directives for script, music, voice, visuals, and captions. This is achieved, for example, by mapping user goals to motivational genres and instructional methods using a concept graph. The functionality of MSG 310 is facilitated by the Emotion State Vector (ESV), which informs the tone, pacing, and emotional framing of each time-coded segment.

Content template repository 312 is a saved set of predefined templates such as breakout/breakdown interludes, guided-meditation flows, mnemonic explainers, and content magnification transitions (e.g., summary-to-detail). These templates are used by the MSG to provide a structured framework for generating personalized content, ensuring that the output is coherent and aligns with established motivational or educational patterns. Content template repository 312 may be updated or modified by reinforcement learning based on user feedback, such as user segment selections or rejections, and statistical trend detection based on actual user behavior. This allows the system to learn which templates are most effective, e.g., for different users and contexts, in order to improve the personalization and engagement of the generated content.

The output of orchestration module 308 is a time-coded modality script, which includes segment roles, directives for various modalities (script, music, voice, visuals, and captions), and bindings to licensed or user-provided resources. This modality script is used by downstream module(s) to coordinate the generation of synchronized multimedia content.

A model agnostic adapter layer (MAAL) 314 receives the modality script and generates normalized media artifacts by dispatching tasks to one or more different AI engines and converting model-specific outputs into a normalized media artifact schema used by downstream module(s). In detail, the algorithm of MAAL includes (a) receiving time-coded segment directives from the MSG 310 (e.g., motivational narration for 30 seconds at a tempo of 90 BPM using a calm mentor voice), then (b) dispatching tasks to the AI engine(s) to generate each segment, and (c) converting model-specific outputs into a normalized artifact schema. For example, a normalized artifact may include audio with a WAV file and phoneme timestamps, music with stems, a beat grid, and BPM, and video with frames, frame presentation timestamps, and resolution. Normalization ensures the system can measure alignment and drift across modalities, and for example, control drift to be less than a threshold value. In some examples, MAAL 314 may utilize the Model Context Protocol (MCP) to interface with AI engines by sharing prompts, embeddings, or contextual data between engines such as a ChatGPT™ model or the like for script generation, a Suno™ model or the like for music generation (but with narration section functionality and song/narration structures), and a Runway™ model or the like for video generation, or may implement equivalent application programming interfaces (APIs) for sharing prompts, embeddings, or contextual data between engines. Generative AI models used by MAAL 314 may include commercial models, such as Eleven Labs or other models with expressive voices, as well as suitable open-source models.

A synchronization and assembly module 316 receives the normalized media artifacts from MAAL 314 and aligns audio, lyrics, vocals, visuals, and interaction cues in real time based on personalization and environmental factors. Module 316 includes a synchronization engine and database 318 (referred to as the SyncGraph) as well as a dynamic media formatter (DMF) 320. SyncGraph 318 may include any suitable alignment representation configured to link beats/bars, phonemes/syllables, caption tokens, video frames, and interaction cues at a desired resolution (e.g., greater than or equal to 10 ms). In particular, the algorithm of SyncGraph 318 includes computing alignment confidence and enforcing drift bounds.

Synchronization and assembly module 316 is configured to ensure the various modalities of the generated content are aligned in time and play cohesively. Module 316 receives normalized media artifacts, which are standardized outputs, ensuring that regardless of the specific AI engine used to generate the content (be it music, narration, or visuals), the system can work with a consistent data structure.

SyncGraph 318 is configured to link different elements across modalities. This linkage occurs at a high resolution, ensuring precise alignment between, for example, the phonemes in a narration, the beats in music, the appearance of captions, and the display of video frames. The SyncGraph computes alignment confidence, providing a metric for how well the different modalities are synchronized. It also enforces drift bounds, ensuring that the modalities do not drift out of sync by more than a specified amount.

DMF 320 may include any suitable media assembly and refinement tool configured to assemble drift-bounded media artifacts and apply bounded repair policies. DMF 320 is utilized to maintain inter-modal drift (e.g., of less than or equal to 50 ms), and functions to apply repair policies such as caption retiming, micro tempo-stretching, layer thinning, and frame resampling. DMF 320 facilitates adaptive streaming and cross-device handoff. The output of module 316 includes a synchronized streaming package with timed metadata.

DMF 320 takes the synchronized elements from the SyncGraph and assembles them into a final presentation. It applies repair policies to correct any misalignments that may occur. These policies can include retiming captions, micro-stretching the tempo of the audio, thinning unnecessary layers, and resampling frames. By applying these repair policies, the DMF ensures that the final output adheres to the specified drift bounds and maintains a high level of synchronization. The DMF also facilitates adaptive streaming and cross-device handoff, adjusting the output to suit different network conditions and device capabilities while preserving synchronization.

The final output of the synchronization and assembly module is a synchronized streaming package that includes timed metadata, such as WebVTT/SRT captions and beat markers, enabling downstream systems to maintain synchronization and adapt the content as needed.

MAAL 314 may also provide output to an intelligence, translation, and quality assurance (QA) module 322 for ensuring the generated content is of high quality, is culturally sensitive, and adapts to the user's evolving needs. Module 322 may include an emotion-preserving translator 324 configured to convert motivational, wellness, educational, or spiritual content into other languages and cultural contexts while conserving tone, prosody, and synchronization; an AI content reviewer 326 configured to perform pre-/post-publish analysis of generated works, creating machine-readable composition/structure/lyric maps and quality summaries; and an adaptive learning and feedback component 328 configured to monitor explicit and implicit signals, as well as optional biometric deltas, and update personalization and MSG templates via reinforcement and statistical trend detection.

More specifically, emotion-preserving translator 324 may include any suitable dual-attention model(s) with quality assurance configured to translate multimedia while preserving semantics, prosody, and emotional impact, and is utilized to provide multilingual adaptation with prosody/tone preservation when content needs to be adapted for different languages or cultural contexts. The algorithm of translator 324 includes (a) extracting sentiment/prosody embeddings (pitch, pauses, emphasis), capturing cultural metadata (region, idioms, formality), and (b) mapping motivational/educational concepts to cultural equivalents via a concept graph. The output of emotion-preserving translator 324 includes translated scripts, captions, and lyric sheets, as well as re-synthesized narration/singing with preserved prosody, and is provided to the Dynamic Media Formatter (DMF) for integration into the final multimedia output.

AI content reviewer 326 may include any suitable software (e.g., a large language model) configured to analyze generated works and generate machine-readable composition/structure/lyric maps and quality summaries of those works. These are utilized to inform section-level recommendations and MSG edits without regenerating entire works when a generated work is assessed for quality and structural integrity. The output of AI content reviewer 326 includes structure maps, alignment confidence, and structure/lyric maps. The output is provided to the Modality Script Generator (MSG) to inform adjustments and refinements.

Adaptive learning and feedback component 328 may include any suitable software configured to provide reinforcement and statistical trend detection, to monitor explicit and implicit signals and optional biometric deltas, and to update personalization and MSG templates when user interactions indicate areas for improvement or adjustment. The algorithm of reviewer 326 may include computing section-level impact scores from interactions with time decay and applying statistical trend detection. The output of adaptive learning and feedback component 328 includes updated ESV priors and MSG templates, and is provided to the personalization agent and MSG to influence future content generation.

Distribution module 330 includes a streaming layer 332 and is configured to package and deliver the final audio/audiovisual outputs using the timed metadata, DMF, and SyncGraph provided by the AI-driven content generation pipeline. In some examples, streaming layer 332 provides a real-time, dynamic, and continuously generated stream of media content. Streaming layer 332 may include any suitable adaptive bitrate (ABR) streaming protocol configured to package the content for delivery to private, community, or branded libraries, with optional forensic watermarking for branded/enterprise use. The output of module 330 comprises synchronized streaming packages with timed metadata (e.g., WebVTT, SRT, beat markers), and is provided to a user device or player application 334, a content delivery network (CDN)/edge delivery system 336, and/or a digital rights management (DRM) and license server 338. CDN/edge delivery system 336 is configured to support offline or edge deployments. In such use cases, the system may pre-cache stems and synchronize with cloud services using directive-only updates, in order to preserve continuity under constrained connectivity. The DRM and license server 338 may be configured to support the monetization models, such as subscription tiers, ad-supported access, enterprise licensing, and blockchain-based royalty splits.

### B. Illustrative Data Flow Diagram

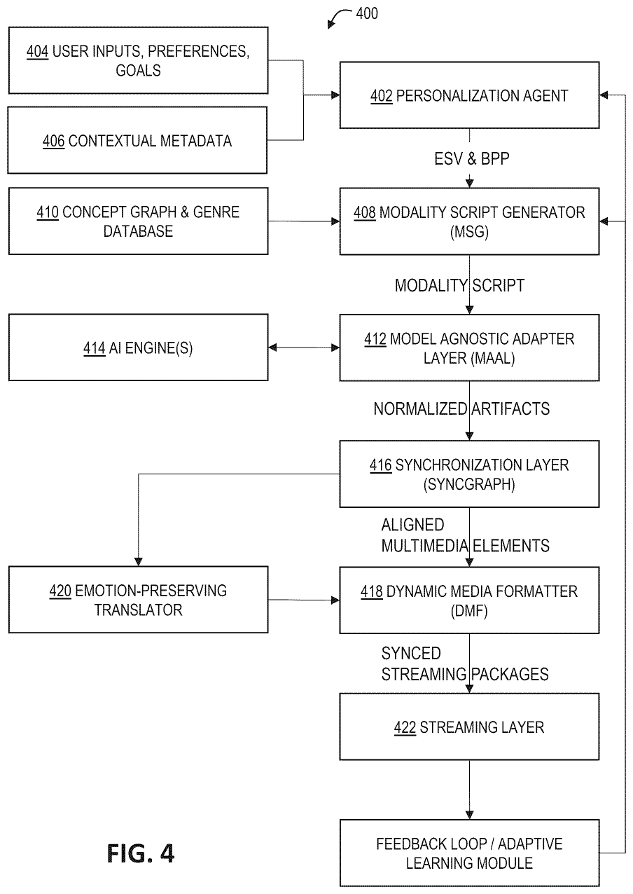

This section describes steps of an illustrative data flow diagram 400 for providing personalized content to a user; see FIG. 4. Aspects of the systems and methods described above may be utilized in the system and method described below. Where appropriate, reference may be made to components and systems that may be used in carrying out each step. These references are for illustration, and are not intended to limit the possible ways of carrying out any particular step or component of the flow diagram. Diagram 400 is an example combining aspects of the systems and methods described above.

A personalization agent 402 receives user inputs, preferences, and goals 404 and contextual metadata 406, and generates or updates an Emotional State Vector (ESV) and Baseline Personality Profile (BPP), e.g., using the methods and algorithms described above. As discussed, the ESV is a numerical vector representing the user's affective state (e.g., current affective state) and the BPP is a vector or set of values indicating the user's stable traits such as preferences, goals, long-term challenges, and persona style. The various inputs going into the ESV and BPP may be received from various sources, such as natural language understanding of user goal statements, sentiment classifiers, wearable biometric sensors (heart rate, skin conductance, posture, gaze), and contextual data such as location, time, and past interaction patterns. The ESV and BPP may be updated or refreshed on a regular basis or cadence. In some examples, the BPP is updated less frequently than the ESV, due to the lower probability of rapid changes in baseline preferences as compared with affective state. For example, while the ESV might update multiple times per second or minute to reflect real-time emotional changes like increased arousal or decreased confidence, the BPP might only be updated when a user explicitly changes a preference setting or completes a new long-term goal. In some examples, ESV refresh rate (e.g., frequency) is user selectable (e.g., qualitatively such as “often”, or quantitatively such as “X times per minute”). In some examples, the BPP includes various settings, some or all of which may be utilized to adjust or affect downstream processes. In some examples, the BPP includes one or more of goals, preferred modalities, avoidance level, tolerance level, time windows, device preferences, genre and reading preferences (e.g., favorite books), and sensitivity. For example, one aspect of the BPP may be a discrete preference for male, female, or neutral voices, such that the text-to-speech adapter within the Model Agnostic Adapter Layer (MAAL) will always be instructed to generate narration audio using the preferred voice gender.

Algorithmically, personalization agent 402 may include software code configured to:
(0) Initialize priors for the ESV based on the BPP, providing a baseline for the user's emotional and contextual state;
(1) Receive explicit (e.g., text or speech) and sensed (e.g., biometric, visual) inputs;
(2) Perform linguistic analysis (e.g., natural language processing NLP) as needed to interpret text or speech inputs;
(3) Perform biometric data processing as needed on raw biometric signals to derive quantitative and meaningful metrics;
(4) Calculate one or more normalized scalar values from 0 to 1, each reflecting a dimension of concern in the ESV, such as arousal, valence, focus, etc., and weighting the different dimensions if desired based on the use case (see further details above);
(5) Construct the ESV based on the normalized scalar values-or- update the ESV based on the latest normalized scalar values, e.g., using exponential smoothing and a hysteresis threshold.

Personalization agent 402 provides the ESV and BPP to downstream aspects of the system, such as a modality script generator 408. Modality script generator (MSG) 408 utilizes various inputs, such as ESV, BPP, and a concept graph and genre database 410. The MSG may include any suitable software configured to generate a modality script, which is a time-coded orchestration plan that specifies segment roles and directives for music, narration, visuals, and captions.

To carry out this function, the MSG uses the Concept Graph and Genre Database in tandem with the ESV and BPP to map user goals and challenges to appropriate modalities and framing methods. The Concept Graph facilitates linking user goals to motivational genres and instructional methods. The MSG produces a time-coded orchestration plan with segment roles, directives, and bindings. The output of the MSG may be based on predefined templates such as breakout/breakdown interludes, guided-meditation flows, mnemonic explainers, and summary-to-detail content transitions.

The modality script may be described as a user-visible and section-editable orchestration plan. The script is designed to be flexible, allowing for parallel segment variants (e.g., mentor vs. coach persona) that can be dynamically switched without full regeneration. The MSG specifies segment roles and directives for script, music, voice, visuals, and captions with bindings to licensed or user-provided resources. The time-coded orchestration plan specifies segment roles and directives for music, narration, visuals, and captions.

The goal of the MSG is to provide downstream modules with a time-coded orchestration plan to facilitate the generation of personalized multimedia content. The MSG acts as a fixed, interactive, or dynamic “screenplay” for AI outputs. Benefits or advantages of this approach may include the ability to dynamically adapt content to a user's emotional state, personal challenges, learning goals, and/or contextual environment. This system supports real-time, personalized, and synchronized content creation that adapts to emotional, physiological, and contextual factors. In some examples, the MSG also supports predefined templates and is user-visible and section-editable, allowing for localized edits without regenerating the entire work.

An illustrative modality script includes the following features: segment roles (intro, verse, chorus, bridge, outro), directives for script, music, visuals, and captions, bindings to licensed or user-provided resources, and support for parallel segment variants (e.g., mentor vs. coach persona). The script may include confidence-building affirmations and references to the user's strengths.

Algorithmically, MSG 408 may include software code configured to:
(0) Determine the user's current emotional and contextual state based on the ESV and retrieve the user's stable traits and preferences based on the BPP.
(1) Map the user's goals to select modalities. For each goal extracted from the user inputs, the Concept Graph is consulted to map the goal to relevant motivational genres, instructional methods, and delivery personas. For example, “test anxiety” might map to “CBT +spaced repetition”. The Genre Database provides templates and stylistic guidelines for the selected motivational genres. The Policy/Constraint Graph may be consulted to ensure that the selected modalities and content sources comply with licensing, provenance, and user consent constraints. A “role” is assigned to each segment (e.g., intro, verse, narration_1, chorus, bridge, narration_2, outro).
(2) Compute segment timing. For example, for each segment, the start and end times may be computed based on the ESV readiness value and a goal duration specified by the user. Segment times may be adjusted to align with natural breaks in the content or to coincide with specific events or milestones.
(3) Generate directives for each segment. Directives include any suitable data structure specifying parameters such as music genre and tempo, narrative beats, voice tone and prosody, caption emphasis, visual style, etc. Directives may be generated for script, music, voice, visuals, and captions, for example based on the ESV arousal and valence values. For example:
   If valence<0.4, empathetic phrasing is used.
   If arousal>0.7, a higher tempo is selected for the music.
   The tone may be determined by a lookup based on valence, e.g., resulting in one of supportive, neutral, or energetic.
(4) Bind resources to the segments. Licensed and/or user-provided resources are bound to the segments based on the directives and the Policy/Constraint Graph. This may involve selecting specific music tracks, narration voices, visual assets, or caption styles.
(5) (Optional) Maintain parallel segment variants. The algorithm may maintain parallel segment variants with different personas or delivery styles (e.g., mentor vs. coach), such that these variants can be switched dynamically without full regeneration, e.g., based on stress or context signals. In some examples, all such operations are logged in the Transform Manifest.
(6) (Optional) Generate dual caption streams comprising a verbatim transcript and supportive overlays. The supportive overlays can be synchronized to cue points to reinforce progress without disrupting pacing.
(7) (Optional) Apply a template to one or more of the segments. Predefined templates may be applied to the segments, such as breakout/breakdown interludes, guided-meditation flows, mnemonic explainers, or content magnification transitions.

The algorithm is configured to output the Modality Script, which comprises a list of time-coded segments, each with a role, directives, and bound resources.

MSG 408 provides the Modality Script to downstream aspects of the system for further processing, such as a model-agnostic adapter layer (MAAL) 412, which is in communication with one or more artificial intelligence engines 414. MAAL 412 serves as an intermediary, enabling different AI engines to work together within the disclosed system for generating personalized multimedia content.

MAAL 412 may include any suitable software configured to act as a translator and unifier to allow different AI engines to operate together to execute the Modality Script. The MAAL provides standardized input/output adapters such that the orchestration plan embodied in the Modality Script executes consistently regardless of which model vendor or library is used. To accomplish this, MAAL 412 is in communication with AI engine(s) 414, which are heterogeneous engines configured to generate content based on the directives provided by the MAAL. For example, AI engine(s) 414 may include the following types of engine for the following purposes or illustrative use cases:
TTS (text-to-speech) for script/narration/voice cloning
Music generation (original, licensed, or remixed)
Visual generation (GAN/diffusion/AR/VR), e.g., for displaying progress gauges or meters, scoring, etc. in XR devices

AI engines 414 may include any suitable artificial intelligence function or service, and may include transformers, diffusion models, or symbolic AI. MAAL 412 is configured to take into account various time-coded segment directives from the Modality Script (e.g., motivational narration for 30 seconds at a tempo of 90 beats per minute (BPM) using a calm mentor voice then change narration voice to “high intensity coach” narration and repeat/loop that section) and generate normalized artifacts accordingly. Specifically, this means the MAAL dispatches tasks to one or more different AI engines depending on the task and converts model-specific outputs into a normalized media artifact schema used by downstream processes. Normalized media artifacts may include audio files with phoneme timestamps, music stems with beat grids and BPM, and video frames with timestamps and resolution. The MAAL may implement Model Context Protocol (MCP) or equivalent APIs for sharing prompts, embeddings, or contextual data between engines. The MAAL functions to ensure the overall system is model-agnostic, provides a single orchestration interface, enables synchronization and editing, and supports mixed deployments.

Algorithmically, MAAL 412 may include software code configured to:
(0) Receive segment directives as part of the Modality Script. The MAAL receives time-coded segment directives from the MSG, which acts as the orchestration plan. These directives may include instructions regarding the type of content to be generated, the desired duration, repeated sections, tempo, and the persona to be used.
(1) Identify the appropriate AI Engine for each modality. Based on the segment directives, the MAAL identifies the specific AI engine needed for each modality, which may involve determining which engine is best suited for tasks like text-to-speech (TTS), music generation, or visual generation. Determining may be accomplished using a lookup table or any other suitable method.
(2) Dispatch tasks to different AI Engines via adapters. The MAAL uses specific adapters to communicate, where each adapter is configured to translate the directives into a format understandable by each AI engine. These may include:
   TTS Adapter: Generates narration audio and phoneme timings from a script.
   Music Adapter: Generates a background track and beat grid.
   Visual Adapter: Generates video frames and timestamps.

Adapters may be unnecessary in cases where the AI engine is able to understand or interpret natively.
(3) Convert AI model-specific outputs into a normalized media artifact schema. Each AI engine produces outputs in its own unique format, so the MAAL converts these into a standardized schema. This normalization enables downstream processes to accurately measure and control alignment and drift across different modalities. For example, standardized schema may include:
   Audio: {wav_file, phoneme_timestamps}
   Music: {stems[ ], beat_grid[ ], bpm}
   Video: {frames[ ], frame_pts[ ], resolution}
(4) Ensure intellectual property rights compliance. The MAAL ensures that all generated content adheres to rights and licensing policies. This involves verifying that licensed and AI-generated elements are compliant with the Policy/Constraint Graph and that all operations are logged in the Transform Manifest.

In summary, the MAAL algorithm standardizes the interaction between different AI engines, enforces rights compliance, and ensures synchronization across modalities, enabling the creation of personalized multimedia content. By normalizing inputs and outputs, the MAAL allows the system to be model-agnostic, meaning that any compatible AI engine can be integrated.

MAAL 412 works in conjunction with a synchronization layer 416, which includes a synchronization graph (AKA SyncGraph), and a dynamic media formatter (DMF) 418 to ensure that all modalities are properly synchronized before output. The SyncGraph may include any suitable software configured to ensure timing, emphasis, and accessibility settings reflect the user's current emotional state, while the DMF may include any suitable software configured to ensure a drift-bounded presentation.

More specifically, the SyncGraph is a representation of the alignment between different elements in the multimedia content, linking beats/bars, phonemes/syllables, caption tokens, video frames, and interaction cues at a selected resolution, e.g., with cross-fading and transitions. The SyncGraph functions to maintain an acceptable inter-modal drift and to provide timing data to other parts of the process. The SyncGraph enables real-time multimodal alignment and drift control, going beyond audio-only prompt generators. It helps to ensure the various components of the multimedia presentation are synchronized, contributing to a cohesive and high-quality user experience. Without the SyncGraph, the different modalities might become misaligned, leading to a disjointed and less effective presentation. Furthermore, the SyncGraph enables further functionality, such as user-facing modification controls (e.g., buttons, sliders, etc.) to repeat, replace, or extend sections.

DMF 418 includes software configured to assemble the final audio-only and/or audiovisual outputs. The DMF functions to apply bounded repair policies such as caption retiming, micro tempo-stretching (e.g., ±1-3%), layer thinning, and frame resampling. The DMF may be configured to apply accessibility settings without breaking SyncGraph timing. The DMF ensures that the final output is polished and accessible, correcting any timing errors and optimizing the presentation for different users and devices, contributing to a high-quality user experience.

In some examples, an Emotion-Preserving Translator 420 translates the multimedia content (scripts, captions, lyrics) into other languages and cultural contexts, with the goal of maintaining the original tone, prosody, and emotional impact of the content. Translator 420 works in tandem with the SyncGraph and DMF to ensure a cohesive output.

Algorithmically, the SyncGraph and DMF may collectively include software code configured to:
(0) Initialize a multigraph data structure configured to represent relationships between different media elements at various points in time. The algorithm receives inputs such as beat grids for music, phoneme timings for vocals, caption timings, and video frames. These inputs are utilized to establish links within the graph.
(1) Create nodes and perform temporal alignment. The algorithm iterates through the timeline of the multimedia content at a high resolution (e.g., 10 ms resolution). For each time point t within the content's duration, it identifies the nearest corresponding media elements. Specifically, for each time t, the algorithm may identify:
   The nearest beat in the music.
   The nearest phoneme in the vocal track.
   The nearest caption to be displayed.
   The nearest video frame.

These identified media elements become nodes in the multigraph, associated with the specific time point t.
(2) Create edges and compute confidence. After identifying the relevant nodes for a given time t, the algorithm creates edges to link the nodes together. Each edge represents the temporal relationship between the media elements. The algorithm computes an alignment confidence score for each edge. This score reflects the certainty that the linked media elements are correctly synchronized. Factors that could influence confidence may include proximity of each element to the time t; accuracy of the beat grid or phoneme timings; and semantic relevance of the caption to the current frame or vocalization.
(3) Enforce drift boundaries. The algorithm enforces a bounded drift, ensuring that media elements remain synchronized within a specified tolerance (e.g., ≤50 ms). The algorithm accomplishes this by checking for drift violations by examining the temporal distances between linked nodes. If the drift exceeds the allowed bound, the algorithm invokes repair policies.
(4) Invoke repair policies as needed. To correct drift violations, the algorithm employs various repair policies, including:
   Caption Retiming: Adjusting the timing of captions to better align with the spoken words or on-screen action.
   Micro Tempo-Stretching: Slightly altering the tempo of the music or speech to compress or expand the timing of a segment. Micro-tempo stretching may be bounded (e.g., ≤±2% or ≤±3%) to avoid noticeable distortion.
   Layer Thinning: Removing non-critical visual elements or audio layers to reduce the complexity of the scene and improve perceived synchronization.
   Frame Resampling: Adjusting the frame rate of the video to better match the audio and maintain synchronization.

The selection and application of repair policies may be based on alignment-confidence thresholds. For example, caption retiming might be applied when the alignment confidence is below a certain level, while more aggressive techniques like tempo stretching are reserved for more severe violations.
(5) Iterate to refine the output. In some examples, the algorithm operates iteratively, repeatedly evaluating and adjusting the synchronization of media elements. This iterative process allows the algorithm to refine the alignment over time, responding to dynamic changes in the user's emotional state or context. As the user interacts with the content, feedback signals (e.g., explicit ratings, saves, repeats, edits, skips, or implicit biometric or camera data) are fed back into the system. This feedback is used to update the Emotion State Vector (ESV), which in turn modulates the Modality Script Generator (MSG) and influences subsequent synchronization decisions.

Once the synchronization meets acceptability criteria, the algorithm outputs a synchronized multimedia presentation, packaging it with timed metadata. This metadata may include things like WebVTT or SRT captions, beat markers for music, timestamps for video frames, and SyncGraph cues.

The SyncGraph and DMF provide synchronized and formatted multimedia outputs to a streaming layer 422. In some examples, the system functions as a real-time adaptive, multimedia streaming service (e.g., “Motivational Multimedia as a Service”) configured to continuously deliver synchronized audio, video, and/or caption streams generated via the ESV→MSG→MAAL→SyncGraph→DMF pipeline. Streaming layer (422) packages time-coded data for playback, adaptive-bit-rate delivery, and provides feedback-loop integration to update the ESV based on user interactions.

Streaming layer 422 functions to deliver multimedia content to the user, and comprises a process of packaging audio and audiovisual outputs with timed metadata. Streaming layer 422 may manage distribution, recommendation, and monetization of the generated content. Accordingly, streaming layer 422 may include any suitable private, community, or branded libraries configured to manage distribution, recommendation, and/or monetization of generated content. For example, streaming layer 422 may function to package content for delivery with DRM/forensic watermarking, provide semantic recommendation using ESV embeddings and Concept Graph mappings, enforce rights via Policy Graph and Transform Manifest, and offer monetization through subscriptions, ad-supported access, enterprise licensing, or blockchain-based royalty splits.

Streaming layer 422 handles tasks such as packaging the content for different devices and network conditions. The streaming layer also ensures that the content is delivered securely, protecting the rights of content creators and distributors. Streaming layer 422 is configured to manage the distribution of personalized content, ensure rights enforcement, and provide opportunities for monetization if desired. In some embodiments, streaming layer integrates real-time personalization, SyncGraph-timed metadata, and manifest-based rights enforcement; proactively delivers content; supports unified playback; enables export to third parties; and/or preserves SyncGraph timing during export.

In some examples, the streaming layer can provide personalized content recommendations to users based on their preferences and viewing habits. This can be done using ESV embeddings, Concept Graph mappings, and engagement telemetry. In some examples, the streaming layer is also responsible for monetizing the content. It can offer different subscription tiers, ad-supported access, and enterprise licensing. Blockchain-based royalty splits can also be implemented to ensure that content creators are fairly compensated.

### C. Use Cases and Embodiments

As outlined below, the systems and methods of the present disclosure may be integrated into various embodiments and utilized in various use cases. Some of these are laid out for illustrative purposes.

### Nano Embodiment Example

In low-resource deployments, the system may provide a nano on-device path. This path maintains a one-second buffer of cached stems and reverts to directive-only playback if network latency exceeds 500 ms, with a hysteresis period of 2-5 seconds. The Nano embodiment operates within approximately 256 MB RAM and a 1 GHz CPU using a lightweight SyncGraph (20 ms resolution) buffering up to 10 seconds of media. Resynchronization on reconnect is performed via a delta-merge algorithm to maintain acceptable drift (e.g., ≤50 ms or ≤100 ms). Edge deployment is supported using distilled models and offline pre-caching up to 500 MB. These numerical values are for illustrative purposes only, and are intended to explain how the system may be implemented in a lower-resource environment if desired. Actual specifications will naturally change over time as the relevant technology evolves.

### Wearable Device Examples

In some embodiments, biometric sensors integrated into wearable devices (e.g., heart-rate monitors, stress sensors, galvanic skin response sensors) may be utilized to collect physiological data such as heart rate, stress levels, and skin conductance. These inputs are applied to adapt content in real time. Biometric deltas may update components of the Emotion State Vector (e.g., arousal, valence, focus), which in turn modulate narration prosody, musical tempo, and visual intensity. Repetition-or cadence-linked sessions may synchronize music pacing to detected movement rates (e.g., running cadence or pace).

Wearable devices may include input and/or output devices or functionality, such as one or more microphone, camera, speakers, computer overlays, computer vision and recognition, voice control, and/or hand or other gesture controls.

### AR/VR/XR Glasses or Headsets

In some examples, augmented-reality (AR) glasses, virtual-reality (VR) headsets, or other extended-reality (XR) systems may be equipped with AI components and integrated with the motivational, educational, and wellness platform of the present disclosure to deliver immersive multimodal experiences. For example, the system may be configured to overlay affirmations, captions, lyrics, or contextual visualizations into the user's environment or field of view during workouts, relaxation sessions, or learning activities.

Each image frame generated by the AR/VR/XR device may be timestamped and bound to SyncGraph nodes, ensuring that captions, narration, musical beats, and visual overlays remain temporally aligned even during head motion or scene transitions. The device's display or optical waveguide serves as the visual output interface for synchronized textual or graphical elements, such as motivational phrases, progress gauges, learning prompts, or animated imagery, presented in harmony with audio playback.

### AI Cameras, Microphones, and Sensors

In some examples, AI-enabled cameras, microphones, and/or sensors in a wearable device are configured to collect multimodal contextual information. For example, cameras may analyze posture, gestures, or exercise repetitions; microphones may capture voice tone and breathing cadence; accelerometers and gyroscopes may provide motion and orientation data; and environmental or biometric sensors may detect lighting, temperature, heart rate, or stress levels. These sensed inputs are utilized to populate or update the ESV. Detected events (e.g., repetition completion, voice inflection changes, motion peaks) may trigger Modality Script cues such as “encourage”, “breathe”, or “final push”.

The DMF may insert supportive captions, images, or overlays at emotional or contextual peaks. The system may employ gesture or voice controls for interaction, enabling users to initiate or modify motivational or educational sequences hands-free.

### Fitness Equipment Integration

The disclosed system may integrate with exercise equipment (e.g., treadmills, resistance machines) to monitor telemetry such as pace, incline, and resistance. These signals can modulate narration cadence or meter, musical tempo, or visual pacing via ESV-driven parameters in the Modality Script. In some embodiments, AI vision systems count repetitions and align tempo or cadence to detected movement rates. All adjustments and events are logged to the Transform Manifest for provenance.

### Illustrative Use Case Examples

a. A user preparing for a public speaking engagement inputs an anxiety state and goal (“deliver a confident speech”). The system generates a motivational narrative aligned with CBT framing and calm-mentor pacing. The script includes confidence-building affirmations, references to the user's strengths, and is delivered with synchronized music and visuals.
b. A user schedules a personalized morning routine that includes motivational messages, energizing music, goal reminders, progress indicators, and visual affirmations. The system generates content overnight, incorporating expected weather and calendar events. The orchestration maintains affect consistency (e.g., key/tempo, voice prosody, visual palette) with ESV guidance. Interaction signals (explicit/implicit) feed the recommendation engine, which computes next best step from section-level impact, short-term trends, and Concept Graph mappings, logging updates in the Transform Manifest.
c. An athlete requests a high-energy audiovisual playlist matching workout intensity. The system adjusts tempo to running pace and inserts motivational prompts at intervals.
d. A student struggling with calculus receives customized content with visualizations and interactive simulations. Instruction adapts to practice-problem performance and biometric stress indicators, increasing focus on concepts where the student exhibits difficulty.
e. For a family road trip, the agent blends motivational and educational segments into an individualized playlist per member, adjusting to time-of-day and travel progress.
f. Wearable-glasses and Headset Use Case

In this example, the electronic device comprises wearable glasses or a headset including one or more image sensors, microphones, motion sensors, and audio-output transducers. The device captures multimodal data in real time to generate or update the ESV. The MSG operates locally on the device and/or communicates with a cloud service to produce time-coded orchestration for audio, captions, and visual overlays.

The SyncGraph enforces a quantified drift bound between narration phonemes, musical beats, caption tokens, and video frames. When alignment confidence falls below a defined threshold, the DMF applies bounded repair policies such as caption retiming, micro-tempo stretching, or frame resampling to preserve perceptual continuity. Generated outputs are rendered to the user through the device's audio transducers (e.g., open-ear, bone-conduction, or enclosed speakers) and, where available, a transparent or opaque micro-display integrated into the headset or glasses.

In this illustrative use case, the system delivers a “context-adaptive motivational coaching” session. While a user walks through an airport after a long flight, the device analyzes camera and microphone data to infer fatigue and low arousal. In response, the platform generates a personalized motivational soundtrack and synchronized overlay, displaying affirmations and tempo-matched visual rhythms to encourage movement and positive focus. User interaction and biometric feedback (e.g., motion recovery, posture, speech tone) dynamically refresh the ESV, allowing real-time adaptation of media elements.

This example demonstrates how the disclosed system enables immersive, emotionally adaptive AR/VR/XR experiences by coupling multimodal sensing, AI-based inference, and synchronized audiovisual generation within wearable computing environments.
g. Public-Speaking/Performance Coaching via Glasses or Headset

In this example, a wearable device may function as a real-time speech and performance coach. Using microphones, cameras, and motion sensors, the device analyzes prosody, pacing, cadence, speaking volume, articulation clarity, posture, facial expression, and gestures. Detected deviations from target delivery parameters are compared to personalized profiles or mentor exemplars stored in the ESV and MSG. The SyncGraph links each analysis frame to active narration segments, allowing the DMF to issue real-time feedback overlays through the display within the bounded drift limit.

Feedback appears as gauges, meters, color bars, or textual prompts such as “Speak louder,” “Slow cadence,” “To target,” “Adjust hand movement,” or “Repeat this section.” Each cue is time-coded and stored in the Transform Manifest with acoustic and motion metrics for post-session analytics. Performance indicators (vocal-energy gauge, gesture-amplitude meter, pacing graph) update continuously to enable quantitative self-correction and longitudinal tracking.

Optional biofeedback visualizations (heart rate, breathing rhythm, stress index) may be displayed for composure training and pacing control.
h. Debating/Argumentation Training and Structured Speech Coaching

In some examples, the system provides debate and argumentation training to develop critical-thinking and persuasive-communication skills. Using the same sensing framework, the system monitors speech tempo, clarity, emphasis, logical structure, and rebuttal timing during live or simulated debates. The ESV tracks cognitive load, composure, and engagement while the MSG structures argument trees or response outlines drawn from curated debate-strategy datasets or licensed educational materials.

During sessions, the DMF renders real-time overlays such as “define term,” “support claim,” “summarize point,” or “pause before rebuttal,” synchronized to presentation timing via the SyncGraph within the bounded drift limit. Post-session analytics evaluate clarity, reasoning coherence, time-management, and audience-response scores, logging results and adaptive improvements in the Transform Manifest.

In collaborative contexts, multiple users may participate through connected headsets or AR environments, with cohort vectors balancing speaking-time fairness and engagement metrics for team debate formats.
i. Audience Attention/Engagement Intelligence

In this example, front-and rear-facing cameras and microphone arrays capture audience and environmental cues during live presentations. The system is configured to analyze facial expressions, body language, applause, laughter, silence, gaze direction, and device distraction (e.g., audience members looking at phones) to estimate collective attention, emotional valence, and engagement intensity.

Aggregated engagement vectors update the ESV in real time. The MSG adjusts narration cadence, emphasis, or projection based on engagement trends (e.g., energize when attention drops, pause on laughter). Using the SyncGraph, audience-event timestamps align with delivery timing; the DMF injects synchronized overlays such as “Maintain eye contact” or “Project voice left.”

Attention heat-maps or gaze-distribution overlays may be rendered post-session to show focus zones, eye-contact ratios, and attention decay. All engagement data and adaptive responses are logged in the Transform Manifest for analytics and reinforcement learning.
j. Post-Performance Replay/Iterative Training

In some examples, captured sessions may be replayed in AR/VR or standard view for analysis. For example, the system may be configured to reconstruct synchronized timelines of speaker delivery, audience reactions, and coaching cues from SyncGraph metadata. Users can view, for example, attention heat maps, engagement curves, vocal-energy graphs, pacing accuracy, and gesture metrics. The DMF may be configured to enable slow-motion and section-specific playback without breaking drift alignment. In some examples, a virtual audience may be re-synthesized from logged affect vectors for realistic rehearsal under varying scenarios.

Performance improvements, timing precision, and audience-connection metrics are recorded to update recommendation weights and personalization profiles for future sessions.
k. Social-Interaction and Communication-Confidence Coaching

In some examples, the system functions as a social-interaction trainer or conversation-confidence coach for users experiencing social anxiety, communication challenges, or spectrum-related difficulties. Using multimodal sensing and adaptive orchestration, the system detects speech tone, pacing, facial expression, eye-contact frequency, and gesture synchrony during live or simulated conversations. The ESV encodes social-comfort parameters (e.g., stress, hesitation, engagement) derived from voice and facial analysis and biometric signals. The MSG retrieves and formats conversation prompts, social-skills techniques, and behavioral exemplars from a curated Social-Guidance Library, including materials from psychology, etiquette, or communication-training sources.

Generated prompts may include small-talk starters (“what I did over the weekend,” “shared interests”), perspective-taking questions, or confidence-building affirmations synchronized to the user's state and context. During sessions, the DMF delivers real-time visual or textual overlays such as “maintain eye contact,” “ask a follow-up question,” “pause and breathe.” For AR/VR use, micro-displays or optical overlays may present interactive coaching cues or dialogue scaffolds aligned to conversation timelines via the SyncGraph. Post-session analytics evaluate reciprocity, speech balance, and comfort progression, producing metrics and personalized next-step exercises logged in the Transform Manifest. Anonymized interaction data may train reinforcement-learning models to suggest progressively complex social scenarios, helping users generalize communication skills to real-world contexts.
l. Other Use Cases
   Future Visualization/Aspirational Projection: Generates future-state visualizations (fitness, posture, skill mastery) via GAN/diffusion/transformer models aligned to ESV goals and bound to SyncGraph events.
   Faith-Based/Spiritual Content: Integrates faith-based narration, chanting, or guided prayer with ESV-adaptive tone and cultural sensitivity; Policy/Constraint Graph enforces rights and provenance.
   Interactive Gamification (Quizzes/Testing): Embeds quiz prompts at SyncGraph nodes; responses adjust MSG segments and ProgressGraph scores; DMF renders timed feedback overlays.
   Enterprise Training/Adaptive Marketing: Delivers role-specific onboarding, compliance explainers, or personalized marketing; outputs manifest-logged and exportable with SyncGraph timing.
   Exercise, Dance, Martial Arts Modules: Detects motion and form via camera or wearables; SyncGraph aligns music/narration; DMF applies bounded repair and provides real-time corrective feedback.
   Conflict Resolution & Localization in Translation: Collaborative editing and translation include automatic conflict resolution (OT/CRDT) and regional localization with emotion-preserving back-translation; all logged in Manifest.

### Motivational Modalities

The system supports a wide range of motivational and behavioral framing techniques. Each modality may be applied singly or in blended combinations (e.g., CBT+Hero's Journey). Modalities may include:
Inspirational: Encourages perseverance and optimism.
Cognitive Behavioral Techniques (CBT): Reframes negative thought patterns into positive behaviors.
Spiritual Guidance: Draws on spiritual or philosophical teachings for mindfulness and inner peace.
Creative Visualization: Guides users through mental imagery for success.
Empowerment & Confidence: Builds self-esteem and assertiveness.
Mindset Shift: Promotes transition from negative to growth-oriented outlook.
Gratitude & Mindfulness: Cultivates present-moment awareness and appreciation.
Problem-Solving & Resilience: Provides strategies for overcoming obstacles.
Achievement & Goal-Setting: Focuses on discipline and long-term objectives.
Self-Compassion & Healing: Promotes emotional healing and acceptance.
Overcoming Loss: Offers support for grief and recovery.
Facing Setbacks: Reinforces resilience in response to challenges.
Addiction Recovery: Provides structure and motivational reinforcement for overcoming addiction.
Conflict Resolution: Teaches empathy and emotional intelligence in disputes.
Overcoming Fear & Anxiety: Supplies practical tools for stress management.
Legacy & Purpose: Inspires purposeful living and legacy building.
Mastery of Time: Reinforces discipline and prioritization.
Overcoming Failure: Positions failure as a learning step toward success.
Energy & Vitality: Elevates energy across physical and mental domains.
Extreme Ownership: Encourages accountability and responsibility.
Mental Toughness: Develops resilience to endure hardship.
The Hero's Journey: Frames user goals as transformational narratives.
Philosophy of Flow: Guides users toward optimal focus states.
Vision & Long-Term Thinking: Promotes forward-looking strategy.
Love & Connection: Reinforces empathy and relational growth.
Purpose-Driven Life: Encourages alignment with spiritual or higher goals.
Action-Based Motivation: Prioritizes immediate, results-oriented behaviors.
High-Performance Habits: Establishes routines for peak performance.
Growth Mindset: Promotes continuous development.
Servant Leadership: Encourages service-based empowerment of others.
Overcoming Self-Doubt: Strengthens belief in one's capabilities.
Dealing with Rejection: Provides strategies for resilience in rejection.
Self-Discipline: Reinforces consistency and control.
Emotional Resilience: Builds capacity to handle stress and setbacks.
Positive Thinking: Encourages optimism and constructive outlook.
Comedic/Dramatization: Uses humor or dramatization to lighten difficult subjects, alleviate anxiety, and improve retention. Both generic and licensed comedic styles may be integrated.

### D. Illustrative Combinations and Additional Examples

This section describes additional aspects and features of personalized content systems, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.
A1. A computer-implemented method, comprising:
   automatically constructing an emotion state vector (ESV) configured to encode an affective state of a user, wherein the ESV comprises a plurality of dimensions each based on a respective sensed input related to the user;
   constructing, based on the ESV, a modality script comprising information defining and orchestrating a plurality of media content items;
   generating the plurality of media content items using one or more generative artificial intelligence (AI) engines;
   arranging the plurality of media content items into a media stream according to the modality script; and
   queueing the media stream to enable playback on an electronic device of the user.
A2. The method of A1, further comprising:
   receiving a baseline personalization profile (BPP) based on stated preferences of the user;
   wherein the modality script is further based on the BPP.
A3. The method of A1 or A0, wherein the dimensions of the ESV are each normalized to be a number from 0 to 1.
A4. The method of any one of A1 through A3, wherein the dimensions of the ESV include one or more dimensions selected from the list consisting of arousal, valence, focus, fatigue, confidence, and readiness.
A5. The method of any one of A1 through A4, wherein the dimensions of the ESV are given different weights depending on a selected use case.
A6. The method of any one of A1 through A5, further comprising:
   normalizing the plurality of generated media content items before arranging the media content items.
A7. The method of A6, wherein arranging the plurality of media content items into the media stream comprises synchronizing the normalized media artifacts to produce a playable media stream comprising aligned media artifacts.
A8. The method of any one of A1 through A7, further comprising:
   causing the media stream to play on the device of the user.
A9. The method of any one of A1 through A8, wherein at least one dimension of the ESV is determined based on a sensed biometric input from a wearable device.
A10. The method of any one of A1 through A9, further comprising:
   dynamically refreshing the ESV in response to receiving a new value for at least one of the sensed inputs.
A11. The method of any one of A1 through A10, wherein the ESV is refreshed at a selected rate greater than twice per second.
A12. The method of any one of A1 through A11, wherein the modality script comprises directions to utilize one or more modalities selected from the list consisting of music, narration, text, image, and video.
A13. The method of any one of A1 through A12, wherein the modality script comprises directions to ensure at least two modalities play simultaneously in at least one segment of the script.
B1. a system for generating personalized digital content, the system comprising:
   one or more processors;
   a memory; and
   a plurality of instructions stored in the memory, wherein the plurality of instructions, when executed by the one or more processors, are configured to:
   automatically construct an emotion state vector (ESV) configured to encode an affective state of a user, wherein the ESV comprises a plurality of dimensions each based on a respective sensed input related to the user;
   construct, based on the ESV, a modality script comprising information defining and orchestrating a plurality of media content items;
   generate the plurality of media content items by communicating with one or more generative artificial intelligence (AI) engines;
   arrange the plurality of media content items into a media stream according to the modality script; and
   queue the media stream to enable playback on an electronic device of the user. B2. The system of B1, wherein the plurality of instructions are further configured to:
   receive a baseline personalization profile (BPP) based on stated preferences of the user;
   wherein the modality script is further based on the BPP.
B3. The system of B1 or B2, wherein the plurality of instructions are further configured to normalize each of the dimensions of the ESV to be a number from 0 to 1.
B4. The system of any one of B1 through B3, wherein the dimensions of the ESV are given different weights depending on a selected use case.
B5. The system of any one of B1 through B4, wherein the plurality of instructions are further configured to:
   normalize the plurality of generated media content items before arranging the media content items.
B6. The system of B5, wherein arranging the plurality of media content items into the media stream comprises synchronizing the normalized media artifacts to produce a playable media stream comprising aligned media artifacts.
B7. The system of any one of B1 through B6, wherein the plurality of instructions are further configured to:
   dynamically refresh the ESV in response to receiving a new value for at least one of the sensed inputs.
B8. The system of any one of B1 through B7, wherein at least one dimension of the ESV is determined based on a sensed biometric input from a wearable device.
C1. A non-transitory computer readable storage medium storing instructions that, when executed, cause one or more processors to carry out the steps of the method of any of A1 through A13.
D1. A computer-implemented method for generating personalized multimedia content, the method comprising:
   receiving user input comprising at least one of prompts, diaries, contextual metadata, biometric signals, or user-uploaded media;
   constructing a personalization state vector encoding affective and contextual features from the user input;
   generating a time-coded orchestration plan that defines segment roles and directives for at least voice output;
   executing the orchestration plan across heterogeneous engines via a model-agnostic adapter layer to produce normalized artifacts;
   synchronizing the artifacts using a synchronization graph linking phonemes, beats, caption tokens, and visual frames with a quantified drift bound;
   assembling a synchronized presentation using a formatter that applies repair policies when alignment confidence falls below a threshold; and
   recording provenance and rights data in a transform manifest and delivering the synchronized presentation to a client device.
D2. The method of D1, wherein the personalization state vector is updated at a cadence of 2 Hz to 10 Hz with smoothing or hysteresis.
D3. The method of D1 or D2, wherein the personalization state vector incorporates contextual factors comprising at least time of day, location, device class, or accessibility preferences.
D4. The method of any one of D1 through D3, wherein the orchestration plan comprises parallel segment variants including different personas or delivery styles that are substituted without regenerating non-variant portions.
D5. The method of any one of D1 through D4, wherein the orchestration plan specifies dual caption streams comprising a verbatim transcript and supportive overlays.
D6. The method of any one of D1 through D5, further comprising generating music synchronized to the orchestration plan with dynamic adjustment of tempo, genre, instrumentation, or style.
D7. The method of any one of D1 through D6, wherein the voice output comprises narration, affirmations, or singing generated with controllable prosody, emotional speech synthesis, or accent customization.
D8. The method of D7, wherein the voice output comprises cloned or licensed voices subject to consent tokens recorded in the transform manifest.
D9. The method of any one of D1 through D8, wherein the synchronization graph enforces a drift constraint of less than or equal to 50 milliseconds across modalities.
D10. The method of any one of D1 through D9, wherein the formatter applies repair policies comprising at least one of caption retiming, micro-tempo stretching, or frame resampling.
D11. The method of any one of D1 through D10, wherein repairs are applied based on alignment confidence thresholds including caption retiming below a first threshold, tempo stretching below a second threshold, and layer suppression below a third threshold.
D12. The method of any one of D1 through D11, wherein the transform manifest records, for each operation, at least an operation type, a segment identifier, a timecode, and a license token.
D13. The method of any one of D1 through D12, wherein license rules comprising overlay-only constraints, segment length limits, or transformation restrictions are enforced prior to export.
D14. The method of any one of D1 through D13, further comprising generating multiple candidate orchestration plans or segments for user selection.
D15. The method of D14, wherein a user edits a selected version by modifying narration text, persona, music directives, or segment length, and the edits are incorporated into the orchestration plan as localized updates without regenerating non-edited portions.
D16. The method of any one of D1 through D15, wherein delivery comprises streaming via an adaptive bitrate protocol while preserving synchronization with device latency profiles.
D17. The method of any one of D1 through D16, wherein the orchestration plan specifies duration profiles comprising short-form bursts, medium-length guided works, or long-form presentations.
D18. The method of any one of D1 through D17, wherein generated narration or captions are translated with semantic and prosodic preservation subject to an emotion-consistency similarity threshold of at least 0.8.
E1. A system for generating personalized multimedia content, comprising:
   a personalization agent configured to construct a personalization state vector from user input;
   a script generator configured to generate a time-coded orchestration plan that defines segment roles and directives for at least voice output; (to a selected or predetermined format)
   a model-agnostic adapter layer configured to execute the orchestration plan across heterogeneous engines and return normalized artifacts;
   a synchronization engine configured to maintain a synchronization graph linking phonemes, beats, caption tokens, and visual frames with a quantified drift bound; a dynamic formatter configured to assemble synchronized presentations and apply repair policies when alignment confidence falls below a threshold; and
   a policy and provenance module configured to record operations and rights data in a transform manifest and deliver the synchronized presentation to a client device.
F1. A non-transitory computer-readable medium storing instructions that, when executed by one or more processors, cause the processors to:
   construct a personalization state vector from user input;
   generate a time-coded orchestration plan that defines segment roles and directives for at least voice output;
   execute the orchestration plan via a model-agnostic adapter layer to produce normalized artifacts;
   synchronize the artifacts using a synchronization graph with a quantified drift bound;
   assemble a synchronized presentation using a formatter that applies repair policies when alignment confidence falls below a threshold; and
   record provenance and rights data in a transform manifest while delivering the synchronized presentation to a client device.
G1. Augmented-reality (AR) glasses, virtual-reality (VR) headsets, and other extended-reality (XR) systems may host the disclosed multimodal orchestration platform. The device overlays affirmations, lyrics, captions, gauges, and visualizations within the user's field of view during workouts, presentations, rehearsals, or relaxation sessions. Each frame is timestamped and bound to SyncGraph nodes, ensuring captions, narration, and music remain aligned during head motion or scene transitions. A micro-display or optical waveguide renders synchronized captions, lyrics, or gauges; the DMF applies bounded-drift repair policies to maintain perceptual alignment within a bounded drift (e.g., ≤100 ms).
G2. The wearable device of G1, wherein gesture or voice inputs update the ESV and MSG in real time for adaptive playback.
G3. The wearable device of G0 or G1, wherein the device is configured to interface directly with the SyncGraph and DMF.
H1. A system or method of any other enumerated paragraph A1-G3, configured or further configured to do one or more of the following:
   interface with licensed libraries of characters, influencers, books, podcasts, videos, personas, commercial songs, stems/remixes, and other media forms;
   select assets using retrieval-augmented generation (RAG) and concept mappings, scoring by semantic similarity, affect alignment, and license eligibility;
   update the ESV during playback based on interaction/biometrics;
   utilize hybrid execution, i.e., distributed on-device plus cloud orchestration;
   analyze audience attention and/or engagement and adapt delivery based on the analysis;
   record and replay sessions with synchronized overlays;
   analyze prosody, pacing, volume, posture, gestures and present informational gauges or text prompts (“speak louder,” “slow cadence,” “repeat section”);
   analyze audience attention and engagement, including facial expressions, body language, gaze direction, applause, laughter, silence, etc.
I1: a system or method of any other enumerated paragraph A1-H1, wherein one or more of the following:
   the electronic device comprises a wearable-glasses or headset apparatus including at least one image sensor, at least one microphone, and at least one audio-output transducer, and wherein the sensed inputs used to construct the Emotion State Vector comprise at least one of image data, audio data, location data, or motion data captured by the apparatus.
   the electronic device comprises a wearable-glasses or headset apparatus configured to capture multimodal data and deliver generated media to the user via one or more audio-output transducers.
   the electronic device further comprises a display integrated into the wearable-glasses or headset apparatus, and wherein arranging the plurality of media content items comprises generating synchronized visual elements comprising at least one of lyrics, captions, or images configured for presentation on the display in alignment with audio playback.
   the electronic device comprises a display integrated into the wearable-glasses or headset apparatus and is configured to present visual media synchronized with audio content comprising at least one of lyrics, captions, or images.
   the audio-output transducer comprises an open-ear or bone-conduction speaker, e.g., configured to maintain environmental situational awareness.
   the wearable-glasses or headset apparatus analyzes audience attention and engagement comprising facial expressions, body language, gaze direction, applause, laughter, silence, and device distraction, and updates the ESV to adapt delivery timing and feedback via a Synchronization Graph.
J1. A system or method of any other enumerated paragraph A1-I1, comprising or further comprising one or more of the following:
   updating the ESV during playback based on user-interaction signals or biometric deltas and modifying the Modality Script in response.
   wherein constructing the Modality Script and generating the media content items are performed across a distributed architecture comprising an on-device processor and a cloud service.
   wherein the Synchronization Graph enforces a bounded drift (e.g., ≤50 milliseconds) between modalities and applies bounded-repair policies including caption retiming, tempo stretching, and frame resampling when alignment confidence falls below a threshold.
   recording and replaying the synchronized presentation with overlaid performance and engagement metrics for iterative training.
   wherein the system analyzes prosody, pacing, volume, posture, and gestures and presents real-time gauges or textual prompts such as “speak louder,” “slow cadence,” or “repeat section,” synchronized via the Synchronization Graph.
   wherein the system functions as a social-interaction trainer by analyzing conversational cues including speech tone, pacing, facial expression, eye contact, and gesture synchrony, retrieving conversation prompts from a social-guidance library, and providing real-time feedback overlays and adaptive dialog scaffolding synchronized via the Synchronization Graph.

### Advantages, Features, and Benefits

The different embodiments and examples of the personalized content system described herein provide several advantages over known solutions for providing customized digital media to users. For example, illustrative embodiments and examples described herein allow or enable real-time, personalized, and synchronized content creation that adapts to emotional, physiological, and contextual factors. This automatic and dynamic adaptation to the user's current state is a significant improvement over existing multimedia platforms that primarily rely on static, pre-recorded materials.

Additionally, and among other benefits, illustrative embodiments and examples described herein facilitate the integration of heterogeneous AI engines through a Model-Agnostic Adapter Layer (MAAL). The MAAL allows the system to work with different AI models for text-to-speech, music generation, image creation, and video synthesis, ensuring that the orchestration plan (MSG) can be executed consistently regardless of the specific model vendor or library being used. This model-agnostic approach provides a single orchestration interface for all modalities and enables precise synchronization and editing.

Additionally, and among other benefits, illustrative embodiments and examples described herein enforce real-time multimodal alignment and drift control through a Synchronization Graph (SyncGraph) and Dynamic Media Formatter (DMF) pipeline. The SyncGraph links beats, phonemes, captions, and video frames with computed alignment confidence, while the DMF assembles final audio-only and audiovisual outputs and applies bounded repair policies to maintain inter-modal drift within acceptable limits. This precise synchronization across modalities ensures a cohesive and engaging user experience, particularly in scenarios involving music, narration, and visuals.

Additionally, and among other benefits, illustrative embodiments and examples described herein provide a compliance-aware orchestration framework with rights-logged outputs and enterprise distribution features. A Policy/Constraint Graph manages provenance, licensing, and rights enforcement, while a Transform Manifest logs drift violations and licensing events. This comprehensive approach to rights management and compliance ensures that licensed and AI-generated elements remain compliant, and that creators are appropriately compensated for their work.

No known system or device can perform these functions. However, not all embodiments and examples described herein provide the same advantages or the same degree of advantage.

### Conclusion

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A computer-implemented method, comprising:
automatically constructing an emotion state vector (ESV) configured to encode an affective state of a user, wherein the ESV comprises a plurality of dimensions each based on a respective sensed input related to the user;
constructing, based on the ESV, a modality script comprising information defining and orchestrating a plurality of media content items;
generating the plurality of media content items using one or more generative artificial intelligence (AI) engines;
arranging the plurality of media content items into a media stream according to the modality script; and
queueing the media stream to enable playback on an electronic device of the user.

2. The method of claim 1, further comprising:
receiving a baseline personalization profile (BPP) based on stated preferences of the user;
wherein the modality script is further based on the BPP.

3. The method of claim 1, wherein the dimensions of the ESV are each normalized to be a number from 0 to 1.

4. The method of claim 1, wherein the dimensions of the ESV include one or more dimensions selected from the list consisting of arousal, valence, focus, fatigue, confidence, and readiness.

5. The method of claim 1, wherein the dimensions of the ESV are given different weights depending on a selected use case.

6. The method of claim 1, further comprising:
normalizing the plurality of generated media content items before arranging the media content items.

7. The method of claim 6, wherein arranging the plurality of media content items into the media stream comprises synchronizing the normalized media artifacts to produce a playable media stream comprising aligned media artifacts.

8. The method of claim 1, further comprising:
dynamically refreshing the ESV in response to receiving a new value for at least one of the sensed inputs.

9. The method of claim 1, wherein at least one dimension of the ESV is determined based on a sensed biometric input from a wearable device.

10. A system for generating personalized digital content, the system comprising:
one or more processors;
a memory; and
a plurality of instructions stored in the memory, wherein the plurality of instructions, when executed by the one or more processors, are configured to:
automatically construct an emotion state vector (ESV) configured to encode an affective state of a user, wherein the ESV comprises a plurality of dimensions each based on a respective sensed input related to the user;
construct, based on the ESV, a modality script comprising information defining and orchestrating a plurality of media content items;
generate the plurality of media content items by communicating with one or more generative artificial intelligence (AI) engines;
arrange the plurality of media content items into a media stream according to the modality script; and
queue the media stream to enable playback on an electronic device of the user.

11. The system of claim 10, wherein the plurality of instructions are further configured to:
receive a baseline personalization profile (BPP) based on stated preferences of the user;
wherein the modality script is further based on the BPP.

12. The system of claim 10, wherein the plurality of instructions are further configured to normalize each of the dimensions of the ESV to be a number from 0 to 1.

13. The system of claim 10, wherein the dimensions of the ESV are given different weights depending on a selected use case.

14. The system of claim 10, wherein the plurality of instructions are further configured to:
normalize the plurality of generated media content items before arranging the media content items.

15. The system of claim 14, wherein arranging the plurality of media content items into the media stream comprises synchronizing the normalized media artifacts to produce a playable media stream comprising aligned media artifacts.

16. The system of claim 10, wherein the plurality of instructions are further configured to:
dynamically refresh the ESV in response to receiving a new value for at least one of the sensed inputs.

17. The system of claim 10, wherein at least one dimension of the ESV is determined based on a sensed biometric input from a wearable device.

18. A non-transitory computer readable storage medium storing instructions that, when executed, cause one or more processors to:
automatically construct an emotion state vector (ESV) configured to encode an affective state of a user, wherein the ESV comprises a plurality of dimensions each based on a respective sensed input related to the user;
construct, based on the ESV, a modality script comprising information defining and orchestrating a plurality of media content items;
generate the plurality of media content items by communicating with one or more generative artificial intelligence (AI) engines;
arrange the plurality of media content items into a media stream according to the modality script; and
queue the media stream to enable playback on an electronic device of the user.

19. The storage medium of claim 18, wherein the instructions, when executed, are further configured to cause the one or more processors to normalize each of the dimensions of the ESV to be a number from 0 to 1.

20. The storage medium of claim 18, wherein the instructions, when executed, are further configured to cause the one or more processors to dynamically refresh the ESV in response to receiving a new value for at least one of the sensed inputs.
